(19) United States
(12) Patent Application Publication
(10) Pub. No.: US 2023/0152753 A1
(43) Pub. Date: May 18, 2023

(54) INTERCHANGEABLE SMARTWATCH SYSTEM

(71) Applicant: Lenovo (United States) Inc., Morrisville, NC (US)

(72) Inventors: Dhruvi Suresh Fulfagar, Concord, NC (US); Jonathan Jen-Wei Yu, Raleigh, NC (US); Hallie MacEldowney, Rolesville, NC (US); Rebecca Megan Frankosky, Raleigh, NC (US)

(21) Appl. No.: 17/528,506

(22) Filed: Nov. 17, 2021

(51) Int. Cl. G04G 21/02; A61B 5/00; G04G 17/04

(52) U.S. Cl. CPC G04G 21/025; A61B 5/681; G04G 17/045

(57) ABSTRACT

An interchangeable smartwatch system includes a smartwatch body and a band including one or more sensors. The smartwatch body is removable from the band, which permits a user to continue to collect data, such as the user's heartrate, when the smartwatch body is removed from the band. The smartwatch body can receive a wireless charge when removed from the band, and then transfer a charge to the band when the smartwatch body is reconnected to the band.

### TECHNICAL FIELD

The present disclosure relates to an interchangeable smartwatch system.

### BACKGROUND

Current smartwatches have affordability and upgradeability issues. Specifically, existing smartwatches either come with a display and a band or just a band. The band and the display must be purchased as one unit, making it a big investment and unaffordable for many users. Users do not have the option to upgrade the smartwatch display to a new one if they wish to over the years. Also, smartwatches are simply not designed to be shared.

Current smartwatches have other issues also. Because existing smartwatches with displays are big and bulky, they are not comfortable for sleeping. Consequently, many users simply remove them prior to going to sleep, and this prevents sleep tracking and data collection. Current smartwatches also lack customization options. Specifically, only the band is interchangeable; the smartwatch screen is not interchangeable. With current smartwatches, there are gaps in data tracking because, for example, some schools/colleges prohibit the use of smartwatches with displays during class and examinations, thereby preventing data tracking during school hours. Also, with current smartwatches, data are not tracked when the watch is being charged. There are further damage and durability issues with current smartwatches. Smartwatches with displays are at a risk of damage while doing intensive workouts like weightlifting, kickboxing, etc. Smartwatch displays are furthermore prone to accumulating dirt, and getting wet while working in the kitchen or around water.

The art is therefore in need of a smartwatch system that addresses these issues.

### BRIEF DESCRIPTION OF THE DRAWINGS

FIG. 1 is an illustration of a smartwatch system.

FIG. 2 is another illustration of a smartwatch system.

FIGS. 3A, 3B, and 3C illustrate different uses of a smartwatch system.

FIG. 4 illustrates a smartwatch band.

### DETAILED DESCRIPTION

In the following description, for purposes of explanation, numerous specific details are set forth in order to provide a thorough understanding of the various aspects of different embodiments of the present invention. It will be evident, however, to one skilled in the art that the present invention may be practiced without all the specific details and/or with variations, permutations, and combinations of the various features and elements described herein.

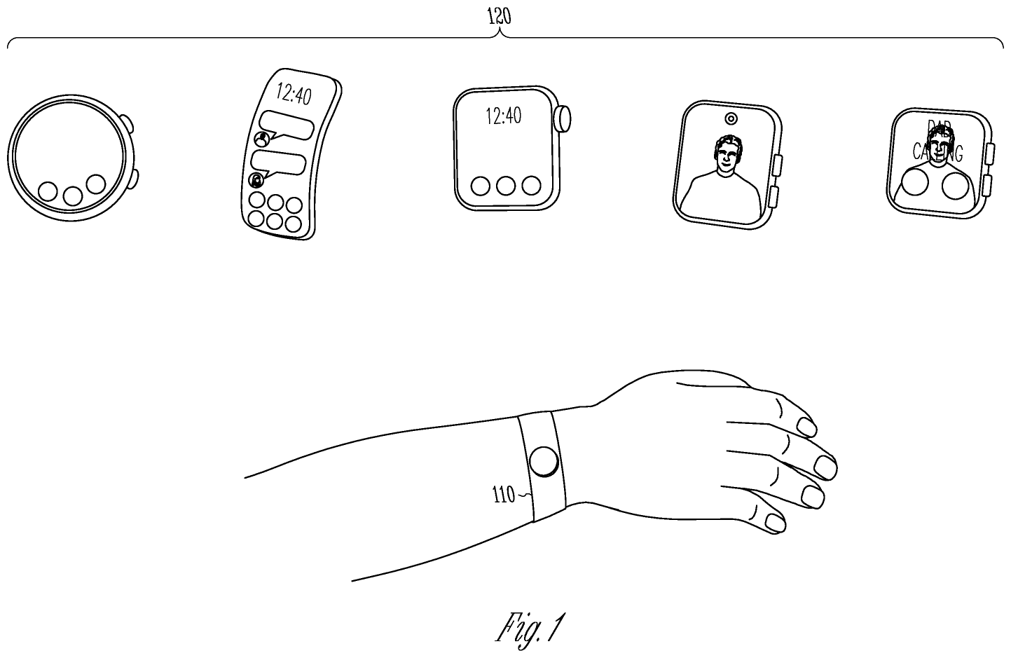

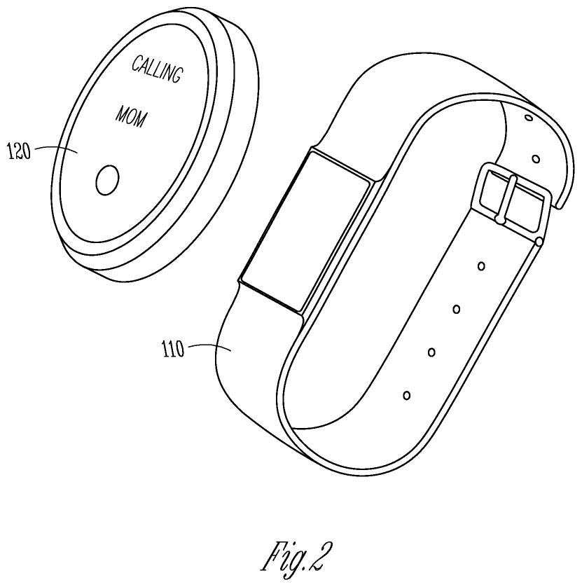

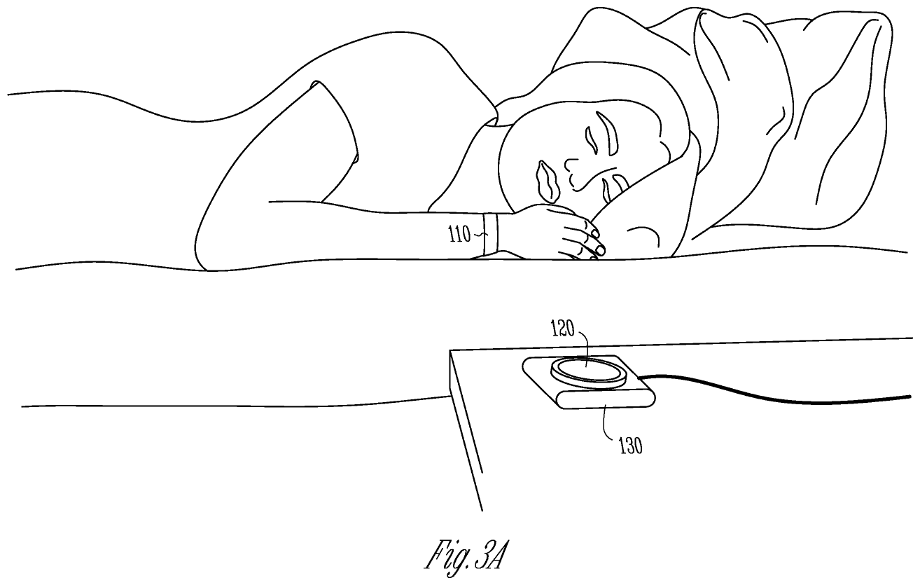

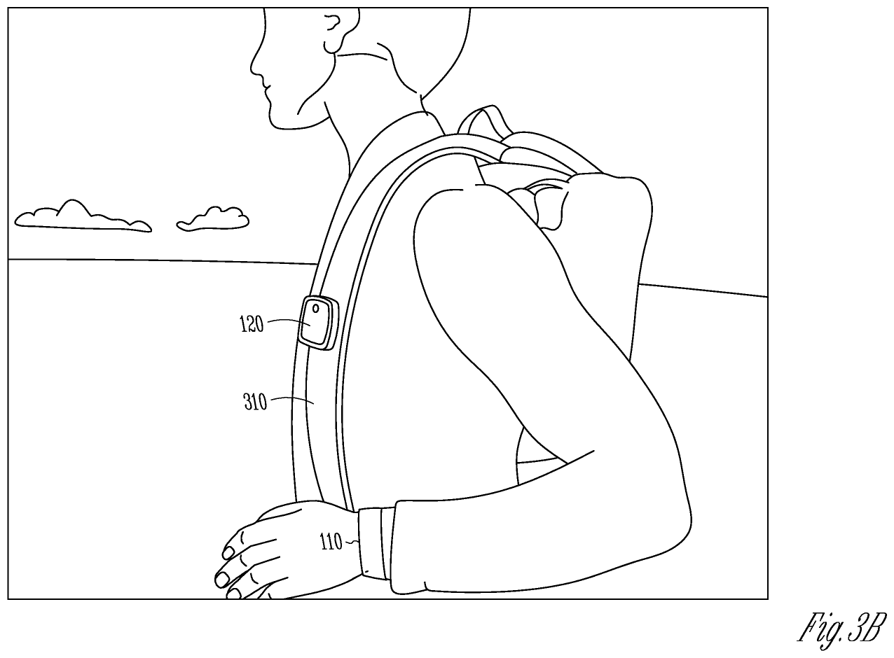

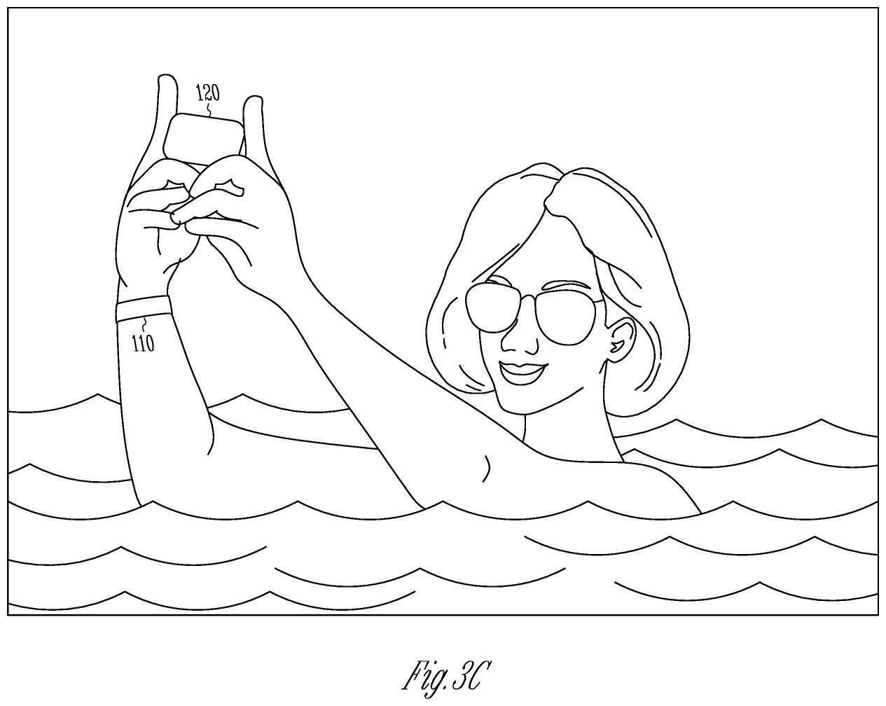

In an embodiment, an interchangeable smartwatch system is a modular smartwatch concept that has two main components. Referring to FIGS. 1 and 2, a first component is the smartwatch band 110. The smartwatch band 110 has embedded sensors in it that track heart rate, oxygen levels in the blood, number of steps, hydration levels, and stress levels as well as sleep. The second component is the smartwatch 120 itself, that is, the smartwatch face, body, or display. As illustrated in FIG. 1, the smartwatch face 120 can include many form factors. The smartwatch face 120 can include a digital screen with an audio device, a microphone, or even a camera (depending on the type of smartwatch face). The smartwatch face 120 can be charged wirelessly (see FIG. 3A), either when it is detached from the band 110 or when it is attached to the band 110. In an embodiment, wireless charging refers to a charger that includes a wired connection for insertion into a typical wall outlet, and a wireless contact point for coupling to the smartwatch face 120 and transferring charged to the smartwatch face 120. The smartwatch face 120 can then transfer charge to the band 110 when it is connected to the band 110. As illustrated in FIGS. 1 and 2, the smartwatch face 120 is interchangeable and can be replaced with other smartwatch displays, for example, another smartwatch face with a camera, a round display, a rectangular display, a minimally small display, and/or a child's version of the display. In an embodiment, the smartwatch face 120 has 5G/LTE connectivity.

Embodiments of the present disclosure permit users to mix and match smartwatch faces 120. Users can own two or more smartwatches, and these users can alternate uses of the smartwatches depending on the users' needs. These embodiments afford a shared family system, assuming easy profile switching. With these embodiments, it is more economical to own multiple smartwatch faces 120 and bands 110.

As noted, embodiments include a removable smartwatch face 120 and a standalone band 110. The removal of the smartwatch face 120 and the wearing of just the band 110 results in a temporarily reduced formfactor while still being able to collect data via the band 110. As illustrated in FIG. 3A, when sleeping, the unwieldy, large formfactor of the smartwatch face 120 acts as a barrier to collecting sleep data, so the smartwatch face 120 is removed from the person and charged by charger 130 while the person sleeps. When exercising or partaking in other leisurely activities such as swimming, many users do not need a display during exercise, and they value the reduced size/weight of only wearing the band 110, especially when swimming, weightlifting, or other exercises where a smartwatch face/display 120 can be problematic or at risk of being damaged. And as illustrated in FIG. 3C, the smartwatch face 120 can be removed in order to be used for other purposes, such as taking a selfie. The removable smartwatch face 120 further provides a “tech break” for the user, such as during family meals, church time, and exams (as noted, many schools forbid smartwatches with displays during exams). Moreover, as discussed in more detail below, a band with a minimal e-ink display could be used to display basic information like heart rate, time, SPO₂ levels, alarms, etc.

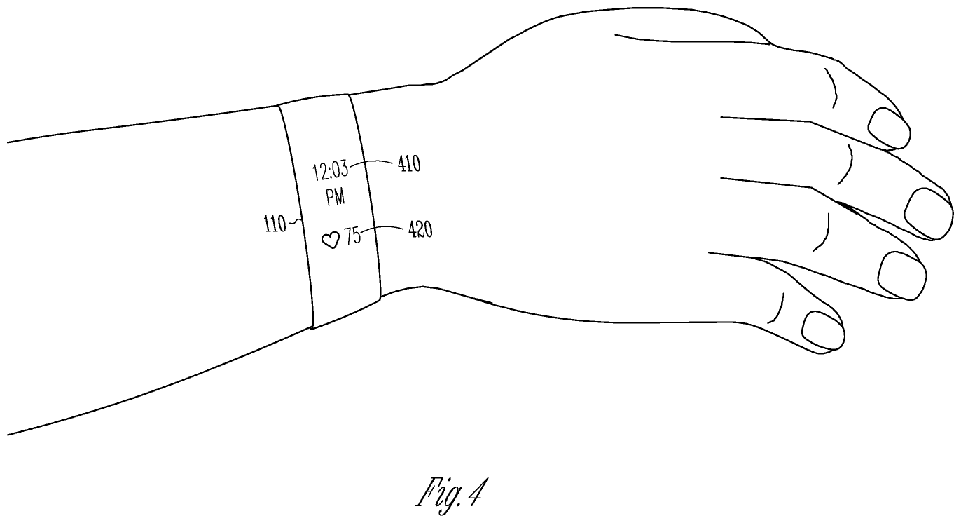

When the smartwatch face 120 is removed from the band 110 for charging and only the band 110 is worn, there are no data gaps because of the continuous wearing of the band 110 and the continuous collection of data by the sensors in the band 110. More specifically, as illustrated in FIG. 3A, the smartwatch face 120 can be charged overnight by charger 130 while the user continues to wear the band 110 and the band 110 continues to track the sleep of the user. In the morning, the smartwatch face 120 is reconnected to the band 110, and the smartwatch face 120 can transfer an electric charge to the band 110. This feature addresses the issue of users who have given up on sleep tracking because of the unwieldy formfactor of the smartwatch face 120. This embodiment also avoids a pain point with current smartwatches, that is, data-gaps caused by the need to charge the smartwatch. Prior to embodiments of the current disclosure, users had to be very conscious to charge their smartwatches during low-activity periods to avoid missing any data collection activity. As illustrated in FIG. 4, these data can be collected by the band 110 while the smartwatch face 120 is detached from the band, and the data can still be displayed to the user via a display on the band, such as display 420 showing the heart rate of the user. The band 110 can also display the time of day 410.

As illustrated in FIG. 3B, the smartwatch face 120 can be attached to an item such as a backpack 310. The smartwatch face 120 can also be attached to pieces of clothing, a bicycle or other helmet, and even a camera that is attached to a person or a person's accessory (such as a GoPro® camera). Of course, the smartwatch face 120 and band 110 can be worn on a person's wrist. The smartwatch face 120 and band 110 can also be worn on a person's ankle, leg, or upper arm.

The modular design of the disclosed embodiments enables a user to build the perfect smartwatch for his or her needs or create an optimized system for his or her family. The ability to change out smartwatch faces gives parents some control over their children's access to technology. Functionality in the band, a public display, and a private display allow for easy adaptation to different uses and personalization.

### EXAMPLES

Example No. 1 is an apparatus that includes a band comprising one or more sensors; and a smartwatch face removably coupled to the band. The smartwatch face is operable for receiving a wireless charge and for transferring a charge to the band. The band can also receive a wireless charge.

Example No. 2 is an apparatus that includes all the features of Example No. 1, and further optionally includes an apparatus wherein the smartwatch face is operable to receive the wireless charge when the smartwatch face is decoupled from the band.

Example No. 3 is an apparatus that includes all the features of Example Nos. 1-2, and further optionally includes an apparatus wherein the one or more sensors are operable to sense data when the smartwatch face is decoupled from the band or the smartwatch is coupled to the band.

Example No. 4 is an apparatus that includes all the features of Example Nos. 1-3, and further optionally includes an apparatus wherein the one or more sensors comprise a heartrate sensor, a blood oxygen level sensor, a step accumulator, a stress level sensor, a sleep sensor, a skin conductance sensor, a global positioning system (GPS) sensor, a hydration sensor, an altimeter, an electrocardiogram (ECG) sensor, a gesture sensor, an ultraviolet (UV) ray sensor, a magnetometer, an electrodermal sensor, a gyroscope, an accelerometers, and a compass.

Example No. 5 is an apparatus that includes all the features of Example Nos. 1-4, and further optionally includes an apparatus wherein the smartwatch face comprises one or more of a digital screen, a microphone, a speaker, and a camera.

Example No. 6 is an apparatus that includes all the features of Example Nos. 1-5, and further optionally includes an apparatus comprising a plurality of smartwatch faces configured for removable coupling to the band.

Example No. 7 is an apparatus that includes all the features of Example Nos. 1-6, and further optionally includes an apparatus wherein one or more of the plurality of smartwatch faces comprise different operating systems. In an embodiment, the different operating systems permit the smartwatch faces to have different features (one smartwatch face may have a camera while another smartwatch face may not), permit the smartwatch face to receive different types of data from the band, and permits the smartwatch face to have other features such as parent operating mode and a child operating mode.

Example No. 8 is an apparatus that includes all the features of Example Nos. 1-7, and further optionally includes an apparatus wherein the band is operable for attachment to one or more of a wrist, an arm, a leg, and an ankle.

Example No. 9 is an apparatus that includes all the features of Example Nos. 1-8, and further optionally includes an apparatus wherein the band is operable for attachment to a helmet, a piece of clothing, a backpack, a purse, and a camera.

Example No. 10 is an apparatus that includes all the features of Example Nos. 1-9, and further optionally includes an apparatus wherein the smartwatch face is operable to collect data from a first band when coupled to the first band, and to collect data from a second band when coupled to the second band (with ease of switching between profiles). In an embodiment, the smartwatch face can treat the data as collected from the same person (who has two or more bands) and/or treat the data as collected from two different persons (who can be identified by data provided by the band).

Example No. 11 is an apparatus that includes all the features of Example Nos. 1-10, and further optionally includes an apparatus wherein the smartwatch face comprises a private function and a public function. In an embodiment for example, the public function could be available to both a parent and child, wherein the private function may only be available to the parent.

Example No. 12 is an apparatus that includes all the features of Example Nos. 1-11, and further optionally includes an apparatus wherein the smartwatch face is coupled to the band via one or more of a magnetic force, an electromagnetic force, and a physical coupling (such as a latch, a buckle, or a Velcro® coupling).

Example No. 13 is an apparatus that includes all the features of Example Nos. 1-12, and further optionally includes an apparatus wherein the band comprises a band display.

Although embodiments have been described with reference to specific examples, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

1. An apparatus comprising:
a band comprising one or more sensors; and
a smartwatch removably coupled to the band;
wherein the smartwatch is operable for receiving a wireless charge and for transferring a charge to the band.

2. The apparatus of claim 1, wherein the smartwatch is operable to receive the wireless charge when the smartwatch is decoupled from the band.

3. The apparatus of claim 1, wherein the one or more sensors are operable to sense data when the smartwatch is decoupled from the band or the smartwatch is coupled to the band.

4. The apparatus of claim 1, wherein the one or more sensors comprise a heartrate sensor, a blood oxygen level sensor, a step accumulator, a stress level sensor, a sleep sensor, a skin conductance sensor, a global positioning system (GPS) sensor, a hydration sensor, an altimeter, an electrocardiogram (ECG) sensor, a gesture sensor, an ultraviolet (UV) ray sensor, a magnetometer, an electrodermal sensor, a gyroscope, an accelerometers, and a compass.

5. The apparatus of claim 1, wherein the smartwatch comprises one or more of a digital screen, a microphone, a speaker, and a camera.

6. The apparatus of claim 1, comprising a plurality of smartwatches configured for removable coupling to the band.

7. The apparatus of claim 6, wherein one or more of the plurality of smartwatches comprise different operating systems.

8. The apparatus of claim 1, wherein the band is operable for attachment to one or more of a wrist, an arm, a leg, and an ankle.

9. The apparatus of claim 1, wherein the band is operable for attachment to a helmet, a piece of clothing, a backpack, a purse, and a camera.

10. The apparatus of claim 1, wherein the smartwatch is operable to collect data from a first band when coupled to the first band, and to collect data from a second band when coupled to the second band.

11. The apparatus of claim 1, wherein the smartwatch comprises a private function and a public function.

12. The apparatus of claim 1, wherein the smartwatch is coupled to the band via one or more of a magnetic force, an electromagnetic force, and a physical coupling.

13. The apparatus of claim 1, wherein the band comprises a band display.

14. An apparatus comprising:
a band comprising one or more sensors and a band display; and
a smartwatch removably coupled to the band;
wherein the smartwatch is operable for receiving a wireless charge when the smartwatch is decoupled from the band and the smartwatch is operable for transferring a charge to the band; and
wherein the one or more sensors are operable to sense data when the smartwatch is decoupled from the band or the smartwatch is coupled to the band.

15. The apparatus of claim 14, wherein the one or more sensors comprise a heartrate sensor, a blood oxygen level sensor, a step accumulator, a stress level sensor, a sleep sensor, a skin conductance sensor, and a global positioning system (GPS) sensor, a hydration sensor, an altimeter, an electrocardiogram (ECG) sensor, a gesture sensor, an ultraviolet (UV) ray sensor, a magnetometer, an electrodermal sensor, a gyroscope, an accelerometers, and a compass.

16. The apparatus of claim 14, wherein the smartwatch comprises one or more of a digital screen, a microphone, a speaker, and a camera.

17. The apparatus of claim 14, comprising a plurality of smartwatches configured for removable coupling to the band.

18. The apparatus of claim 17, wherein one or more of the plurality of smartwatches comprise different operating systems.

19. The apparatus of claim 14, wherein the smartwatch is operable to collect data from a first band when coupled to the first band, and to collect data from a second band when coupled to the second band.

20. The apparatus of claim 14, wherein the smartwatch comprises a private function and a public function.
